(19) United States
(12) Patent Application Publication
(10) Pub. No.: US 2019/0336579 A1
(43) Pub. Date: Nov. 7, 2019

(54) COMPOSITIONS AND METHODS TO INDUCE TARGETED APOPTOSIS BY MODULATING PROTEIN TYROSINE PHOSPHATASE 13 (PTPN13)

(71) Applicant: National Jewish Health, Denver, CO (US)

(72) Inventors: David W.H. Riches, Highlands Ranch, CO (US); Alison Bamberg, Denver, CO (US)

(21) Appl. No.: 16/293,248

(22) Filed: Mar. 5, 2019

Related U.S. Application Data
(63) Continuation of application No. 15/489,475, filed on Apr. 17, 2017, now abandoned.
Application No. 15/489,475 is a continuation of application No. 13/291,838, filed on Nov. 8, 2011, now abandoned.
(60) Provisional application No. 61/411,813, filed on Nov. 9, 2010.

(51) Int. Cl. A61K 38/19; A61K 31/713; A61K 38/20; A61K 38/21

(52) U.S. Cl. CPC A61K 38/191; A61K 31/713; A61K 38/217; A61K 38/208; A61K 38/2006

(57) ABSTRACT

Embodiments herein concern compositions and methods for treating a subject having or suspected of developing a pulmonary disorder or cancer. Certain embodiments concern modulating protein tyrosine phosphatase non-receptor type 13 (PTPN13) expression and/or activity in a subject to treat uncontrolled cellular growth in the subject.

### PRIORITY

The instant application is a continuation application of U.S. patent application Ser. No. 15/489,475, filed Apr. 17, 2017, which is a continuation application of U.S. application Ser. No. 13/291,838 filed Nov. 8, 2011, now abandoned, which claims the benefit of U.S. Provisional Patent Application Ser. No. 61/411,813, filed Nov. 9, 2010. These applications are incorporated herein by reference in their entirety for all purposes.

### STATEMENT REGARDING FEDERALLY FUNDED RESEARCH

This invention was made with Government support under grant 5R01HL068628-09 awarded by the National Institutes of Health. The Government has certain rights in the invention.

### FIELD

Embodiments herein report methods, compositions and uses for modulating apoptosis of cellular populations in a subject. This application also generally reports methods, compositions and uses for targeting fibroblasts in a subject in need thereof. In certain embodiments, the targeted fibroblasts are lung fibroblasts. In other embodiments, compositions and methods herein concern modulating protein phosphatases. In some embodiments, modulating protein phosphatases includes modulating protein tyrosine phosphatase non-receptor type 13 (PTPN13). Other embodiments include compositions and methods for treating a subject having cancer.

### BACKGROUND

Idiopathic interstitial pneumonia (IIP) is a chronic lung disorder characterized by progressive scarring of the alveolar interstitium leading to severe dyspnea, hypoxemia, and death. Idiopathic pulmonary fibrosis (IPF) is a progressive, fatal, fibrotic lung disease of unknown origin that typically leads to death within 5 years of diagnosis. Idiopathic pulmonary fibrosis (IPF) is the most common type of idiopathic interstitial pneumonia (IIP) and has the highest mortality.

### SUMMARY

Embodiments herein report methods, compositions and uses for treatment of uncontrolled growth of cells in a subject. In accordance with these embodiments, targeted uncontrolled growth can include uncontrolled growth of fibrotic cells. In certain embodiments, the uncontrolled cells are lung cells (e.g. fibroblasts and myofibroblasts) due to a pulmonary disorder. In some embodiments, pulmonary disease in a subject can include, but is not limited to, pulmonary fibrosis. Certain embodiments relate to compositions and methods for early intervention of pulmonary disease. Other embodiments concern analyzing certain enzymes known to function in part to modulate apoptosis of various lung cells. In accordance with these embodiments, enzymes include, but are not limited to protein tyrosine phosphatases (PTP). One PTP target of the instant application includes, but is not limited to, protein tyrosine phosphatase non-receptor type 13 (PTPN13).

Some embodiments concern targeting uncontrolled accumulation and/or activation of lung fibroblasts and myofibroblasts in fibroblastic foci. In certain embodiments, impaired apoptosis of these cells can be one factor that facilitates accumulation of these cells. In certain embodiments, compositions that target PTPN13 expression or activity will induce apoptosis in a targeted cell population to reduce accumulation of undesirable cells (e.g. in pulmonary fibrosis, cancer, scarring, wound healing, grafts etc). Some embodiments include compositions and methods for treating a subject having idiopathic pulmonary fibrosis (IPF).

In certain embodiments, PTPN13 activity can include tyrosine phosphatase catalytic activity and protein:protein interactions (e.g. Fas and other signaling molecules). In addition, PTPN13 can also interact with I-kappa-B-alpha. In certain embodiments, methods and compositions disclosed herein are capable of blocking PTPN13 in several ways, including, but not limited to, block PTPN13 expression, block PTPN13 interaction with Fas or other molecules and/or block PTPN13 tyrosine phosphatase catalytic activity.

Other embodiments concern treating a subject having cancer where cancerous cells have become resistant to Fas-induced apoptosis. Cancers can include, but are not limited to pancreatic cancer, stomach cancer, colon cancer, lung cancer, melanoma or other cancers having this trait. Compositions and methods disclosed herein can be used to treat a subject having cancer in order to induce apoptosis in tumor cells of the subject, thereby shrinking the tumor or reducing the number of tumor cells in the subject. Some of these compositions may also inhibit metastasis of tumor cells. It is contemplated herein that the disclosed compositions and methods can be combined with any other compositions or methods known in the art to treat a subject having cancer or other disorder described herein.

Certain embodiments include using an agent that associates with PTPN13 in order to modulate its binding to Fas. Methods and compositions disclosed herein may be used alone or in combination with other treatment methods known in the art directed to induce apoptosis in a cell or cell population.

Other embodiments can include reducing PTPN13 expression or activity in other tissues or anatomical locations in a subject. In accordance with these embodiments, targeted administration by PTPN13 expression modulating agents can be administered to reduce uncontrolled cell growth in a predetermined anatomical region or tissue in the subject. In certain examples, these methods can be used to treat a subject recovering from surgery (e.g. plastic surgery) or a burn, keloids or other wound healing or uncontrolled growth such as cancer (e.g. cancers having uncontrolled cell growth such as a fibrotic tumor and skin graft patients etc.)

Other embodiments reported herein are directed towards methods of treating or preventing pulmonary disorders in a subject. In accordance with these embodiments, a subject having or suspected of developing a pulmonary disorder (e.g. pulmonary fibrosis) may be treated with one or more agents that induce apoptosis of overproduced fibroblasts and/or myofibroblasts. For example, one or more agents may be introduced to a subject that target protein tyrosine phosphatase non-receptor 13 (PTPN13) regulation or expression.

Agents contemplated of use in any embodiments disclosed herein, can include antibodies, aptamers, anti-sense-RNA, siRNAs, small molecules, cytokines (e.g. inflammatory cytokines) or any other agent capable of associating with or binding to and/or affecting PTPN13 expression and/or activity. Certain embodiments described concern downregulation and/or inhibition of PTPN13 expression or activity by agents contemplated herein. In certain embodiments, PTPN13 catalytic activity can be modulated. In accordance with these embodiments, broad spectrum phosphatase inhibitors may be used in compositions described herein or more directed agents can be used to modulate PTPN13 catalytic activity.

### BRIEF DESCRIPTION OF THE DRAWINGS

The following drawings form part of the present specification and are included to further demonstrate certain embodiments of the present invention. The embodiments may be better understood by reference to one or more of these drawings in combination with the detailed description of specific embodiments presented herein.

FIGS. 1A-1D represent exemplary cross-sections of tissue having uncontrolled cell death. FIG. 1A. Negative control, FIG. 1B. Ovarian Cancer, FIG. 1C. Lung tissue from a patient with biopsy proven idiopathic pulmonary fibrosis with a pathology of usual interstitial pneumonia (IPF/UIP) and FIG. 1D. Lung tissue from a patient with cryptogenic organizing pneumonia, a control interstitial lung disease that can be treated with glucocorticoids (COP).

FIGS. 2A-2D represent exemplary experiments where: FIG. 2A. represents in the top panel a quantitative RT-PCR analysis of PTPN13 mRNA; lower panel is a Western blot analysis for PTPN13 protein. FIG. 2B. A time course of PTPN13 mRNA expression following TNF-α (10 ng/ml) and IFN-γ (50 U/ml) treatment. FIG. 2C. A Western blot analysis of PTPN13 expression after MRC5, NHLF, and FHLF treated with TNF-α (10 ng/ml) and IFN-γ (50 U/ml) for times indicated. FIG. 2D. A time course of caspase-8 activation in MRC5 lung fibroblasts, NHLF and FHLF.

FIGS. 3A-3C represent exemplary experiments where: FIG. 3A. represents specific siRNA decreases PTPN13 expression. NHLF, FHLF and MRC5 lung fibroblasts were treated with PTPN13 siRNA (75 mM), scrambled siRNA (75 mM) as a negative control or 50 U/ml IFN-γ and 10 ng/ml TNF-α as a positive control for 36 hours. PTPN13 expression was quantified by western blot analysis and densitometry was performed. FIG. 3B. represents NHLF, FHLF and MRC5 lung fibroblasts were incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α. Fas activating antibody (250 ng/ml) (Fas activating antibody) or PBS (unstimulated) was added for 2 hours before analyzing Caspase-8 activation by a fluorescence-based assay. FIG. 3C. represents NHLF, FHLF and MRC5 lung fibroblasts incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α. Fas activating antibody (250 ng/ml) (Fas activating antibody) or PBS (unstimulated) was added for 2 hours before analyzing Caspase-3 activation by a fluorescence-based assay.

FIGS. 4A-4D represent exemplary experiments where: FIG. 4A. Represents MRC5 lung fibroblasts incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α as a positive control. Co-precipitated caspase-8 and FADD were identified by western blot analysis. FIG. 4B. Represents MRC5 lung fibroblasts incubated in medium (0.1% serum) alone or were treated for 2 hours with acylated-SLV peptide (10 uM) or acylated-SVL peptide (10 uM) as a negative control. Treatment with IFN-γ (50 U/ml) and TNF-α (10 ng/ml) for 36 hours was used as a positive control. Fas activating antibody (250 ng/ml) (Fas activating antibody) or PBS (unstimulated) was then added for 2 hours before analyzing Caspase-8 activation by a fluorescence-based assay. FIG. 4C. Represents MRC5 lung fibroblasts incubated in medium (0.1% serum) alone or were treated with acylated-SLV peptide (10 uM) or acylated-SVL peptide (10 μM) as a negative control for 2 hours. Treatment with IFN-γ (50 U/ml) and TNF-α (10 ng/ml) for 36 hours was used as a positive control. Fas activating antibody (250 ng/ml) (Fas activating antibody) or PBS (unstimulated) was then added for 2 hours before analyzing Caspase-3 activation by a fluorescence-based assay. FIG. 4D. Represents MRC5 lung fibroblasts incubated in medium (0.1% serum) alone or treated with SLV (10 uM) for 2 hours. Treatment with SVL (10 uM) for 2 hours was used as a negative control and 50 U/ml IFN-γ and 10 ng/ml TNF-α for 36 hours was used as a positive control. Co-precipitated caspase-8 and FADD were identified by western blot analysis.

FIGS. 5A-5E represent exemplary experiments where: FIG. 5A. Represents effects of TNF-α and IFN-γ on tyrosine phosphorylation of Fas. MRC5 lung fibroblasts treated with TNF-α (10 ng/ml) and IFN-γ (50 U/ml) for indicated times. Western blot analysis of tyrosine phosphorylation of tyrosine 291 in Fas death domain using a phospho-specific anti-Fas Y291 antibody. FIG. 5B. Represents protein tyrosine phosphatase inhibitor sensitizes lung fibroblasts to Fas-induced apoptosis. MRC5 lung fibroblasts either preincubated with pervanadate (150 uM) or PBS as a control for 2 hours. FIG. 5C. Represents protein tyrosine phosphatase inhibitor increases tyrosine phosphorylation of tyrosine 291 on Fas. MRC5 lung fibroblasts were either pre-incubated with pervanadate (150 uM) or PBS as a control for 2 hours. FIG. 5D. Represents MRC5 lung fibroblasts incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α as a positive control. FIG. 5E Represents MRC5 lung fibroblasts incubated in medium (0.1% serum) alone or were treated with acylated-SLV peptide (10 uM) or acylated-SVL peptide (10 uM) as a negative control for 2 hours.

FIGS. 6A-6J represent exemplary experiments where: FIG. 6A. Represents a survival curve of saline and bleomycin treated wildtype and PTPN13−/− mice. FIG. 6B. Represents measurement of static compliance. FIG. 6C. Represents collagen production measured indirectly by hydroxyproline. The upper right lobe was digested overnight. FIG. 6D. Represents increased cell numbers in BALF. FIG. 6E. Represents differential of BALF. Macrophages, lymphocytes and neutrophils counted from cytospin. FIG. 6F. Represents Capillary leak in murine lungs. Albumin was measured by ELISA present in BALF. FIG. 6G. Represents collagen content as measured by stereology. FIG. 6H. Represent percent disease in wildtype and PTPN13−/− mice treated with bleomycin. FIG. 6I and FIG. 6J represent cross sections of lung of wildtype and PTPN13−/− samples regarding collagen dispersion throughout the representative samples.

### DEFINITIONS

Terms that are not otherwise defined herein are used in accordance with their plain and ordinary meaning.

As used herein, “a” or “an” may mean one or more than one of an item.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known methods or components have not been included in the description.

In accordance with embodiments of the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

Fibrogenesis is mediated in part by the uncontrolled accumulation and activation of lung fibroblasts and myofibroblasts in fibroblastic foci wherein they secrete collagen and other matrix components. In the lungs of IPF subjects, impaired apoptosis is thought to contribute to fibroblast and myofibroblast accumulation. Experiments using in situ labelling of lung tissue from IPF subjects show markers of apoptosis in the epithelium adjacent to the fibroblastic foci, but apoptotic markers are not present in the myofibroblasts themselves, suggesting resistance to apoptosis in the myofibroblasts. Human lung fibroblasts basally express Fas but are resistant to Fas-induced apoptosis. A recent study demonstrated that exposure to TNF-α and IFN-γ prior to Fas ligation overcomes basal resistance. Pretreatment with these cytokines promoted Fas-induced death-inducing signaling complex (DISC) formation, suggesting that these cytokines influenced the expression or activity of an inhibitory protein upstream of FADD in the Fas mediated apoptosis signaling cascade.

PTPN13 is a member of the protein tyrosine phosphatase (PTP) family that is ubiquitously expressed in normal human tissues. PTPN13 associates with the carboxyterminal SLV tripeptide sequence of human Fas and has been implicated in resistance to apoptosis in multiple human cancers. Also, overexpression of PTPN13 has been shown to inhibit the export of Fas to the cell surface. PTPs are known to be signaling molecules that regulate a variety of cellular processes including cell growth, differentiation, mitotic cycle, and oncogenic transformation. PTPN13 is ubiquitously expressed in normal human tissues, including lymph node and peripheral blood mononuclear cells samples. Functionally, the PTPN13 gene has been tested for association to diseases (Bone Neoplasms; Carcinoma; Colorectal Neoplasms; Liver Neoplasms; lymphoma; Sarcoma, Ewing's), proposed to participate in a pathway (FAS signaling pathway (CD95)) and a process (protein tyrosine amino acid dephosphorylation). Proteins are expected to have molecular functions (non-membrane spanning protein tyrosine phosphatase activity, hydrolase activity, protein binding, structural molecule activity) and to localize in various compartments (e.g. nucleus, cytoplasm, cytoskeleton).

In certain embodiments disclosed herein compositions and methods are disclosed for reducing PTPN13 expression or activity in a subject. In accordance with these embodiments, agents capable of reducing PTPN13 expression can be administered to a subject in order to facilitate or induce cell death of unwanted or uncontrolled cells. Some embodiments include but are not limited to, agents such as antibodies, aptamers, anti-sense-RNA, siRNAs, small molecules, cytokines or any other agent that is capable of associating with and/or affecting PTPN13 expression and/or activity. Certain embodiments described concern downregulation and/or inhibition of PTPN13 expression or activity by agents contemplated herein. In certain embodiments, PTPN13 catalytic activity can be modulated. In accordance with these embodiments, broad spectrum phosphatase inhibitors can be used or more directed agents can be used to modulate PTPN13 catalytic activity. In certain embodiments, cytokines including, but not limited to, TNF-α and IFN-γ alone or in combination can be administered in a composition to reduce PTPN13 expression in order to regulate uncontrolled cell growth. In some embodiments, reduced PTPN13 expression can alleviate inhibition of apoptosis in a subject thereby enabling the formation of death-inducing signaling complex (DISC) to induce apoptosis to control unwanted cell death.

In certain embodiments, regulation of PTPN13 expression in lung tissues can be used to treat certain lung conditions. In some embodiments, a lung condition may be an incurable disease. Certain embodiments herein report methods, compositions and uses for treatment of uncontrolled growth of lung cells in a subject. In accordance with these embodiments, targeted uncontrolled growth can include targeting uncontrolled growth of fibrotic cells in lungs of a subject. In certain embodiments, the uncontrolled cells are lung cells (e.g. fibroblasts and myofibroblasts) due to a pulmonary disorder in the subject. In some embodiments, pulmonary disease in a subject can include, but is not limited to, pulmonary fibrosis. Certain embodiments relate to compositions and methods for early intervention of pulmonary disease in a subject having a pulmonary condition. Other embodiments concern controlling expression of certain enzymes known to function in part to modulate apoptosis of various lung cells. In accordance with these embodiments, enzymes include, but are not limited to protein tyrosine phosphatases (PTP). One PTP target of the instant application includes, but is not limited to, protein tyrosine phosphatase non-receptor 13 (PTPN13).

Some embodiments concern targeting uncontrolled accumulation and/or uncontrolled activation of lung fibroblasts and myofibroblasts in fibroblastic foci. Other embodiments concern targeting uncontrolled accumulation and activation of other fibroblast cells such as in wound healing or cosmetic surgery or transplants (e.g. skin grafting in burn victims or other conditions). In certain embodiments, impaired apoptosis of these cells can be one factor that facilitates accumulation of these cells. Some embodiments include compositions and methods for treating a subject having idiopathic pulmonary fibrosis (IPF).

Other embodiments concern treating a subject having cancer where cancerous cells have become resistant to Fas-induced apoptosis. Cancers can include, but are not limited to pancreatic cancer, stomach cancer, ovarian cancer, colon cancer, lung cancer, melanoma or other cancers having this trait. Compositions and methods disclosed herein can be used to treat a subject having cancer in order to induce apoptosis in tumor cells of the subject, thereby reducing the tumor size, inhibiting expansion of the tumor or reducing number of tumor cells in the subject. These compositions and methods can be combined with any other compositions or methods known in the art to treat a subject having cancer. In accordance with these embodiments, a subject can be treated by any method known. In certain embodiments, a subject can be treated by targeting the specific anatomical location(s) of the tumor or administered directly to an affected organ with compositions for reducing expression of PTPN13.

Certain embodiments include using an agent that associates with PTPN13 in order to modulate its binding to Fas in part to induce Fas-receptor induced cell death. Methods and compositions disclosed herein may be used alone or in combination with other treatment methods known in the art directed to induce apoptosis in a cell or cell population or tumor.

Other embodiments can include reducing PTPN13 expression in other tissues in a subject. In accordance with these embodiments, targeted administration by PTPN13 expression modulating agents can be administered to reduce uncontrolled cell growth in a predetermined anatomical region in the subject. In certain examples, these methods can be used to treat a subject recovering from surgery (e.g. plastic surgery) or a burn or other wound healing or uncontrolled growth such as cancer (e.g. cancers having uncontrolled cell growth such as a fibrotic tumor etc.).

In certain embodiments, a pulmonary disorder can include, but is not limited to, pulmonary fibrosis. In accordance with these embodiments, pulmonary fibrosis, can include, but is not limited to, idiopathic pulmonary fibrosis (IPF), familial interstitial pneumonia (FIP) and other pulmonary fibrosis disorders. In some embodiments, one or more samples may be obtained from a subject suspected of having, or with a propensity for developing, a pulmonary disorder including, but not limited to, IPF and FIP. Samples obtained from the subject can be analyzed before and after administering a composition capable of modulating PTPN13. In certain embodiments, one or more samples can be a tissue sample or a fluid sample. In accordance with these embodiments, a sample can be a lung air space sample (e.g. bronchoalveolar lavage (BAL)) or a blood sample, a tissue sample could include a lung biopsy or cells isolated from a biopsy and cultured in vitro. Samples from different sources may be compared to obtain additional information and perform additional comparisons for prognosis and/or early intervention of pulmonary disease in the subject. In some embodiments, a subject can be administered an agent that modulates PTPN13 in order to treat a symptom of pulmonary fibrosis.

Some embodiments concern treating a subject having idiopathic pulmonary fibrosis (IPF). IPF is a fatal lung disease that severely limits the ability of patients to breathe. Two thirds of IPF patients die within 3 years of diagnosis accounting for approximately 45,000 American deaths every year. Unlike breast cancer, from which a similar number of American women die each year, there is no effective pharmacologic or biologic therapy for IPF. IPF is caused by injury to, and aberrant repair of, the lower lung resulting in the accumulation of fibroblasts cells. These cells make abundant amounts of collagen and contribute to the formation of scar tissue (fibrosis) thereby impeding oxygenation of the blood. In normal wound repair, fibroblasts die and are removed at the completion of the repair process. In contrast, fibroblasts persist and continue to accumulate in the lungs of IPF patients leading to progressive and unrelenting fibrosis.

In yet other embodiments, a subject having a pulmonary disorder can be administered additional agents in combination with PTPN13 associating agents, for example, pro-inflammatory cytokines. Pro-inflammatory cytokines can include, but are not limited to, one or more of TNF-α, IFN-γ, IL1β, IL-18, IL-12 and other inflammatory cytokines known in the art. In certain embodiments, a subject having a pulmonary disorder can be administered one or more combinations of TNF-α and IFN-γ. In accordance with these embodiments, PTPN13 mRNA and/or protein expression and activity as defined previously can be modulated by administering to a subject one or more compositions of pro-inflammatory cytokine(s) to modulate PTPN13 expression. In other embodiments, a subject having a pulmonary disorder can be administered one or more inflammatory cytokines to modulate sensitivity of lung fibroblasts to apoptosis or increase apoptosis of unwanted lung fibroblasts in the subject. In accordance with these embodiments, lung fibroblasts of the subject can have increased sensitivity to Fas-induced apoptosis and/or increased caspase activity (e.g. capase-8, caspase 3 etc). In certain embodiments, these treatments can lead to an increase in apoptosis of lung fibroblasts relative to an affected control subject not receiving such a treatment in order to modulate the fibroblasts growth or expansion in the subject. It is contemplated herein that agents that modulate the activity of PTPN13 can affect the symptoms of pulmonary fibrosis and may prolong a subject's life having such a disorder by, for example, reducing fibroblast and/or myofibroblast accumulation.

### Antisense

The term “antisense” is intended to refer to polynucleotide molecules complementary to a portion of a targeted gene or mRNA species. “Complementary” polynucleotides are those that are capable of base pairing according to the standard Watson Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5 methylcytosine, 6 methyladenine, hypoxanthine and others in hybridizing sequences does not interfere with pairing.

Antisense polynucleotides, when introduced into a target cell, specifically bind to their target polynucleotide and interfere with transcription, RNA processing, transport, translation and/or stability. Antisense RNA constructs, or DNA encoding such antisense RNA's, may be employed to bind to a nucleic acid region for identification of that region in vitro or in vivo, such as within a subject, including a human subject. Antisense constructs may be designed to bind to the promoter and other control regions, exons, introns or even exon intron boundaries of a gene.

siRNA

Small interfering RNAs (siRNA) are contemplated for compositions, methods and uses herein. siRNA sometimes known as short interfering RNA or silencing RNA, is a class of double-stranded RNA molecules, about 20-25 nucleotides in length but can be larger or smaller, that play a variety of roles in biology. In certain embodiments, siRNA can be used for RNA interference (RNAi) pathway, where it interferes with the expression of a target gene. In addition to their role in the RNAi pathway, siRNAs also act in RNAi-related pathways, e.g., as an antiviral mechanism or in shaping the chromatin structure of a genome; the complexity of these pathways is only now being elucidated.

### Aptamers

In certain embodiments, molecules may be detected by binding agent of use may be an aptamer. Methods of constructing and determining the binding characteristics of aptamers are well known in the art. For example, such techniques are described in U.S. Pat. Nos. 5,582,981, 5,595,877 and 5,637,459, each incorporated herein by reference.

Aptamers may be prepared by any known method, including, but not limited to, synthetic, recombinant, and purification methods. Aptamers may be used alone or in combination with other ligands for the same target. In general, a minimum of approximately 3 nucleotides, preferably at least 5 nucleotides, are necessary to effect specific binding. Aptamers of sequences shorter than 10 bases may be feasible, although aptamers of 10, 20, 30 or 40 nucleotides may be preferred.

Aptamers may be extended with flanking regions and otherwise derivatized. In a further embodiment, the flanking sequence may comprise a specific sequence that preferentially recognizes or binds a moiety to enhance the immobilization of the aptamer to a substrate.

Aptamers may be isolated, sequenced, and/or amplified or synthesized as conventional DNA or RNA molecules. Alternatively, aptamers of interest may comprise modified oligomers. Any of the hydroxyl groups ordinarily present in aptamers may be replaced by phosphonate groups, phosphate groups, protected by a standard protecting group, or activated to prepare additional linkages to other nucleotides, or may be conjugated to solid supports. One or more phosphodiester linkages may be replaced by alternative linking groups, such as P(O)O replaced by P(O)S, P(O)NR2, P(O)R, P(O)OR′, CO, or CNR2, wherein R is H or alkyl (1-20 C) and R′ is alkyl (1-20 C); in addition, this group may be attached to adjacent nucleotides through O or S. Not all linkages in an oligomer need to be identical.

Aptamers may be single-stranded or double-stranded DNA or RNA. A starting aptamer may contain a randomized sequence portion, generally including from about 10 to 400 nucleotides, or about 20 to 100 nucleotides. Randomized sequences can be flanked by primer sequences that permit the amplification of aptamers found to bind to the target. For synthesis of the randomized regions, mixtures of nucleotides at the positions where randomization is desired may be added during synthesis. Methods for preparation and screening of aptamers that bind to particular target molecules of interest are well known in the art and are contemplated herein.

### Antibodies and Antibody Fragments

Any known methods for developing antibodies or antibody fragments are contemplated herein (e.g. polyclonal, monoclonal, antibody fragments that bind to agents or molecules disclosed herein etc). Antibodies contemplated herein may be used as a treatment and/or diagnosis of pulmonary disease or predictor of propensity for survival in a subject having or suspected of developing a pulmonary disorder. In certain embodiments, PTPN13 antibodies may be used to assess or protein levels and quantitative polymerase chain reaction (qPCR) may be used to assess PTPN13 mRNA in a subject having a pulmonary disorder. It is contemplated herein that antibodies or antibody fragments may be taken up by cells and used to modulate PTPN13 production (e.g. nucleic acid or protein expression) in a subject having or suspected of developing pulmonary fibrosis. In certain embodiments, one or more agents capable of modulating PTPN13 may be used to treat a subject having or suspected of developing a pulmonary disorder.

One or more antibodies or antibody fragments may be generated to associate with PTPN13 disclosed herein by any method known in the art.

### Polypeptide Fragments

One aspect of the invention pertains to isolated proteins, and biologically active portions thereof, as well as polypeptide fragments suitable for use as immunogens to raise antibodies directed against a polypeptide. In one embodiment, the native polypeptide PTPN13 can be isolated from cells or tissue sources by an appropriate purification scheme using standard protein purification techniques. In another embodiment, polypeptides are produced by recombinant DNA techniques (e.g. TNF-α and INF-γ). Alternative to recombinant expression, a polypeptide of the invention can be synthesized chemically using standard peptide synthesis techniques.

### Recombinant Molecules

Recombinant unmodified and mutant variants of proteins contemplated herein can be used in compositions to induce apoptosis in a targeted population. Nucleotide sequences of the proteins may also be produced. These nucleotide sequences may be used as starting material to generate all of the variants and amino acid fragments contemplated herein, using recombinant DNA techniques and methods known to those of skill in the art.

An “isolated” or “purified” protein or biologically active portion thereof is substantially free of cellular material or other contaminating proteins from the cell or tissue source from which the protein is derived, or substantially free of chemical precursors or other chemicals when chemically synthesized. The language “substantially free of cellular material” includes preparations of protein in which the protein is separated from cellular components of the cells from which it is isolated or recombinantly produced. Thus, protein that is substantially free of cellular material includes preparations of protein having less than about 30%, 20%, 10%, or 5% (by dry weight) of heterologous protein (also referred to herein as a “contaminating protein”). When the protein or biologically active portion thereof is recombinantly produced, it is also preferably substantially free of culture medium, i.e., culture medium represents less than about 20%, 10%, or 5% of the volume of the protein preparation. When the protein is produced by chemical synthesis, it is preferably substantially free of chemical precursors or other chemicals, i.e., it is separated from chemical precursors or other chemicals which are involved in the synthesis of the protein. Accordingly such preparations of the protein have less than about 30%, 20%, 10%, 5% (by dry weight) of chemical precursors or compounds other than the polypeptide of interest.

Biologically active portions of a polypeptide (e.g. pro-inflammatory cytokines) of the invention include polypeptides comprising amino acid sequences sufficiently identical to or derived from the amino acid sequence of the protein, which include fewer amino acids than the full length protein, and exhibit at least one activity of the corresponding full-length protein. Typically, biologically active portions comprise a domain or motif with at least one activity of the corresponding protein. A biologically active portion of a protein of the invention can be a polypeptide which is, for example, 10, 20, 25, 40, 50, 100 or more amino acids in length. Moreover, other biologically active portions, in which other regions of the protein are deleted, can be prepared by recombinant techniques and evaluated for one or more of the functional activities of the native form of a polypeptide of the invention.

Embodiments described herein also pertain to variants of the polypeptides contemplated of use to modulate PTPN13 expression. Such variants have an altered amino acid sequence which can function as either agonists (mimetics) or as antagonists. Variants can be generated by mutagenesis, e.g., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein which function as either agonists (mimetics) or as antagonists can be identified by screening combinatorial libraries of mutants, e.g., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (e.g., for phase display). There are a variety of methods which can be used to produce libraries of potential variants of the polypeptides of the invention from a degenerate oligonucleotide sequence. Methods for synthesizing degenerate oligonucleotides are known in the art.

An isolated polypeptide (e.g. PTPN13, TNF-α, IFN-γ), or a fragment thereof, can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (or 10, 15, 20, or 30) amino acid residues of the amino acid sequence and encompasses an epitope of the protein such that an antibody raised against the peptide forms a specific immune complex with the protein.

In each of the aforementioned aspects and embodiments of the invention, fusion polypeptides are also specifically contemplated herein. In one embodiment, fusion polypeptides can be produced by recombinant DNA techniques. Alternative to recombinant expression, a fusion polypeptide of the invention can be synthesized chemically using standard peptide synthesis techniques. Compositions contemplated herein can include fusion polypeptide(s) and a pharmaceutically acceptable carrier, excipient or diluent. In each of the above-recited methods, TNF-α, IFN-γ or other pro-inflammatory cytokines or peptide fragment heterologous to the protein can be used.

### Modes of Administration

Modes of administration of the various therapeutic agents contemplated herein are exemplified below. However, the agents can be delivered by any of a variety of routes including: by injection (e.g., subcutaneous, intramuscular, intravenous, intraarterial, intraperitoneal), by continuous intravenous infusion, intranasally, by inhalation, topically, by drops, intradermally, cutaneously, dermally, transdermally, orally (e.g., tablet, pill, liquid medicine), by implanted osmotic pumps (e.g., Alza Corp.), by suppository or aerosol spray.

Those skilled in the art of biochemical synthesis will recognize that for commercial-scale quantities of peptides, such peptides are preferably prepared using recombinant DNA techniques, synthetic techniques, or chemical derivatization of biologically or chemically synthesized peptides.

Agents contemplated herein of use as therapeutic agents in the treatment of a physiological condition. The peptides may be administered as free peptides or pharmaceutically acceptable salts thereof. The term “pharmaceutically acceptable salt” refers to those acid addition salts or metal complexes of the peptides which do not significantly or adversely affect the therapeutic properties (e.g. efficacy, toxicity, etc.) of the peptides. The peptides should be administered to individuals as a pharmaceutical composition, which, in most cases, will comprise the peptide and/or pharmaceutical salts thereof with a pharmaceutically acceptable carrier. The term “pharmaceutically acceptable carrier” refers to those solid and liquid carriers, which do not significantly or adversely affect the therapeutic properties of the peptides.

In other embodiments, small molecules can be administered to a subject capable of blocking or reducing Fas-PTPN13 interactions and interaction of PTPN13 with other signaling molecules. Small molecules known in the art to promote Fas-dependent apoptosis are contemplated.

Pharmaceutical compositions containing proteins contemplated herein or a functional derivative thereof may be administered to individuals, particularly humans, either intravenously, subcutaneously, intramuscularly, intranasally, orally, topically, transdermally, parenterally, gastrointestinally, transbronchially and transalveolarly. Topical administration is accomplished via a topically applied cream, gel, rinse, etc. containing therapeutically effective amounts. Transdermal administration is accomplished by application of a cream, rinse, gel, etc. capable of allowing agents to penetrate the skin and enter the blood stream. Parenteral routes of administration include, but are not limited to, direct injection such as intravenous, intramuscular, intraperitoneal or subcutaneous injection. Gastrointestinal routes of administration include, but are not limited to, ingestion and rectal. Transbronchial and transalveolar routes of administration include, but are not limited to, inhalation, either via the mouth or intranasally and direct injection into an airway, such as through a tracheotomy, tracheostomy, endotracheal tube, or metered dose or continuous inhaler (nebulizer). In addition, osmotic pumps may be used for administration. The necessary dosage will vary with the particular condition being treated, method of administration and rate of clearance of the molecule from the body.

Although the compounds described herein and/or their derivatives may be administered as the pure agent, the active agent may be administered as a pharmaceutical composition. Therefore, it is contemplated that a pharmaceutical composition can include one or more compounds and/or a pharmaceutically acceptable salt thereof, together with one or more pharmaceutically acceptable carriers and, optionally, other therapeutic and/or prophylactic ingredients are contemplated. The carrier(s) must be acceptable in the sense of being compatible with the other ingredients of the composition and not deleterious to the recipient thereof.

Pharmaceutical compositions include those suitable for intranasal, or parenteral (including intramuscular, subcutaneous, cutaneous, inhaled and intravenous) administration. The compositions may, where appropriate, be conveniently presented in discrete unit dosage forms and may be prepared by any of the methods well known in the art of pharmacy. Such methods include the step of bringing into association the active compound with liquid carriers, solid matrices, semi-solid carriers, finely divided solid carriers or combinations thereof, and then, if necessary, shaping the product into the desired delivery system.

Pharmaceutical compositions suitable for oral administration may be presented as discrete unit dosage forms such as hard or soft gelatin capsules, dissolving microbeads with protein carriers, cachets or tablets, each containing a predetermined amount of the active ingredient; as a powder or as granules; as a solution, a suspension or as an emulsion. The active ingredient may also be presented as a bolus, electuary or paste. Tablets and capsules for oral administration may contain conventional excipients such as binding agents, fillers, lubricants, disintegrants, or wetting agents. The tablets may be coated according to methods well known in the art., e.g., with enteric coatings.

Oral liquid preparations may be in the form of, for example, aqueous or oily suspension, solutions, emulsions, syrups or elixirs, or may be presented as a dry product for constitution with water or another suitable vehicle before use. Such liquid preparations may contain conventional additives such as suspending agents, emulsifying agents, non-aqueous vehicles (which may include edible oils), or preservatives. The compounds may also be formulated for parenteral administration (e.g., by injection, for example, bolus injection or continuous infusion) and may be presented in unit dose form in ampoules, pre-filled syringes, small bolus infusion containers or in multi-dose containers with an added preservative. The compositions may take such forms as suspensions, solutions, or emulsions in oily or aqueous vehicles, and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g., sterile, pyrogen-free water, before use.

For topical administration to the epidermis, the compounds may be formulated as ointments, creams or lotions, or as the active ingredient of a transdermal patch. Suitable transdermal delivery systems are contemplated. Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. At least two types of release are possible in these systems. Release by diffusion occurs when the matrix is non-porous. The pharmaceutically effective compound dissolves in and diffuses through the matrix itself. Release by microporous flow occurs when the pharmaceutically effective compound is transported through a liquid phase in the pores of the matrix.

Compositions suitable for topical administration in the mouth include unit dosage forms such as lozenges comprising active ingredient in a flavored base, usually sucrose and acacia or tragacanth; pastilles comprising the active ingredient in an inert base such as gelatin and glycerin or sucrose and acacia; mucoadherent gels, and mouthwashes comprising the active ingredient in a suitable liquid carrier.

When desired, the above-described compositions can be adapted to provide sustained release of the active ingredient employed, e.g., by combination thereof with certain hydrophilic polymer matrices, microbeads, microspheres or other slow-release and or targeted system e.g., comprising natural gels, synthetic polymer gels or mixtures thereof. Pharmaceutical compositions according to the invention may also contain other adjuvants such as flavorings, coloring, antimicrobial agents, or preservatives.

It will be further appreciated that the amount of the compound, or an active salt or derivative thereof, required for use in treatment will vary not only with the particular salt selected but also with the route of administration, the nature of the condition being treated and the age and condition of the subject and will be selected, ultimately, at the discretion of the attending physician.

A pharmaceutical composition of the invention may contain an appropriate pharmaceutically acceptable carrier as defined supra. These compositions can take the form of solutions, suspensions, tablets, pills, capsules, powders, and sustained-release formulations. Such compositions will contain an effective therapeutic amount of the active compound together with a suitable amount of carrier so as to provide the form for proper administration to the subject.

In general, the compound is conveniently administered in unit dosage form; for example, containing 5 to 2000 mg, conveniently 10 to 1000 mg, most conveniently, 50 to 500 mg of active ingredient per unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations, such as multiple inhalations from an insufflator or by application of a plurality of drops into the eye.

Actual dosage levels of active ingredients in the pharmaceutical compositions of this invention may be varied so as to obtain an amount of the active compound(s) that is effective to achieve the desired therapeutic response for a particular subject, compositions, and mode of administration. The selected dosage level will depend upon the activity of the particular pharmaceutical compound or analogue thereof of the present invention, the route of administration, the severity of the condition being treated, and the condition and prior medical history of the subject being treated. However, it is within the skill of the art to start doses of the pharmaceutical compound at levels lower than required to achieve the desired therapeutic effect and to gradually increase the dosage until the desired effect is achieved.

Pharmaceutical compositions can be used in both veterinary medicine and human therapy. The magnitude of a prophylactic or therapeutic dose of the pharmaceutical composition of the invention in the acute or chronic management of pain associated with above-mentioned diseases or indications will vary with the severity of the condition to be treated and the route of administration. The dose, and perhaps the dose frequency, will also vary according to the age, body weight, and response of the individual subject. In general, the total daily dose range of the pharmaceutical composition of this invention is generally between about 1 to about 100 mg, preferably about 1 to about 20 mg, and more preferably about 1 to about 10 mg of active compound per kilogram of body weight per day are administered to a mammalian subject. If desired, the effective daily dose may be divided into multiple doses for purposes of administration, e.g. two to four separate doses per day.

Actual amounts of the active ingredient will vary with each case, according to the species of mammal, the nature and severity of the particular affliction being treated, and the method of administration. In general, the pharmaceutical compositions of the present invention are periodically administered to subject as necessary to improve symptoms of the particular disease being treated. The length of time during which the compositions are administered and the total dosage will necessarily vary with each case, according to the nature and severity of the particular affliction being treated and the physical condition of the subject or subject receiving such treatment.

Useful dosages of the agents and compositions contemplated herein can be determined by comparing their in vitro activity, and in vivo activity in animal models. Methods for the extrapolation of effective dosages in mice, and other animals, to humans are known to the art.

Some embodiments herein relate to treatment and prognosis of pulmonary disease in a subject. In accordance with these embodiments, a subject may be assessed by methods and compositions disclosed herein alone or in combination with any known methods for treatment or prognosis. In addition, early intervention of the pulmonary disease may be combined with embodiments disclosed herein by treating the subject with any composition disclosed herein (e.g. inflammatory cytokines and other associating agents) or method or composition known in the art to treat pulmonary disorders.

### Kits

In certain embodiments, a kit contemplated herein may include compositions for treating a subject having a pulmonary disorder, recovering from surgery or cancer Contemplated herein are methods, compositions and kits for inducing targeted apoptosis in a subject having excessive fibroblast or myofibroblast production in order to attenuate the condition in a subject.

Kits may include a container or delivery means. Any of the kits will generally include at least one vial, test tube, flask, bottle, syringe or other container means, into which the testing agent, may be preferably and/or suitably aliquoted. Kits herein may also include methods for comparing results such as a suitable control sample, for example a positive and/or negative control. Compositions of use in a kit may include, but are not limited to, one or more inflammatory cytokines, nucleic acid ligands or aptamers, anti-sense molecules, siRNAs, small molecules, antibodies, or antibody fragments or other molecules capable of associating with or affecting PTPN13 expression or activity in a subject.

### EXAMPLES

Examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the embodiments which are disclosed and still obtain a like or similar result without departing from the spirit and scope of the invention.

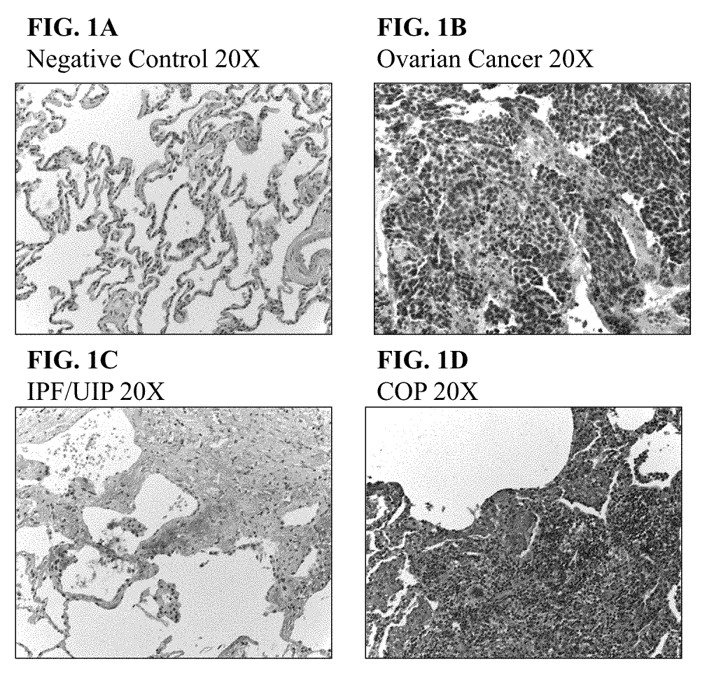

In one exemplary method, to investigate the significance of PTPN13 in idiopathic pulmonary fibrosis and cancer, immunostaining to assess the presence and localization ofPTPN13 was conducted. PTPN13 was weakly detected in alveolar macrophages, but was absent from alveolar epithelial cells in normal lung tissues. PTPN13 was also absent from alveolar epithelial cells in lung tissues from patients with idiopathic pulmonary fibrosis (FIG. 1C) and cryptogenic organizing pneumonia (a control interstitial lung disease that unlike idiopathic pulmonary fibrosis can be treated with glucocorticoids, FIG. 1D). However, PTPN13 was detected in the nuclei and cytoplasm of fibroblasts and myofiboblasts in fibroblastic foci of patients with idiopathic pulmonary fibrosis (FIG. 1C). PTPN13 was also detected in the cytoplasm and nuclei of ovarian cancer cells (FIG. 1B).

### Example 1

### TNF-α and IFN-γ Decrease PTPN13 Expression

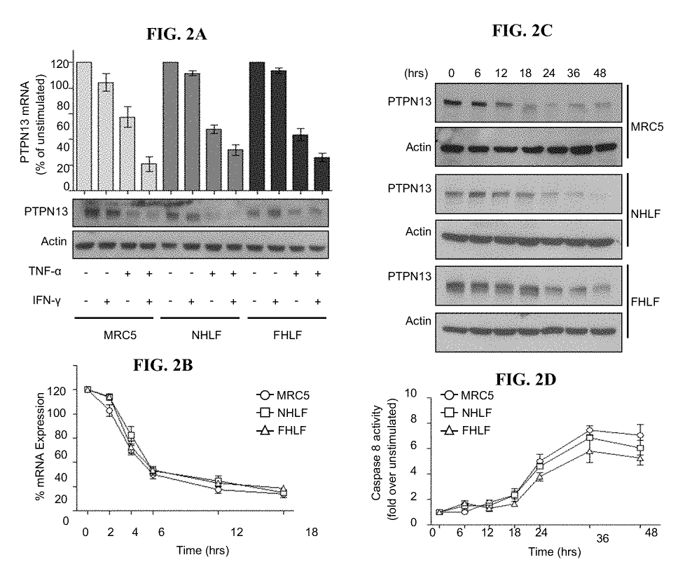

It was previously demonstrated that human lung fibroblasts basally express Fas; however, lung fibroblasts are comparatively resistant to Fas induced apoptosis. Exposure to TNF-α and IFN-γ prior to Fas ligation was demonstrate to overcome this basal resistance, sensitizing the lung fibroblast to Fas-induced apoptosis. To assess the effects of these cytokines on PTPN13 expression, non-diseased normal human lung fibroblasts (NHLF), fibrotic human lung fibroblasts (FHLF) and a fetal lung fibroblast cell line, MRC5 cells, were treated with TNF-α (10/ng/ml), IFN-γ (50 U/ml) or with both cytokines for 6 hours. PTPN13 expression was measured by quantitative PCR (FIG. 2A). The primary lung fibroblasts from normal and fibrotic lung had equivalent levels of PTPN13 expression to the MRC5 lung fibroblast cell line. The NHLF, FHLF and MRC5 cells treated with IFN-γ alone had a minimal decrease in PTPN13 mRNA expression compared to untreated cells. Treatment with TNF-α alone caused a 40% decrease in PTPN13 expression. Combined exposure to TNF-α and IFN-γ decreased PTPN13 mRNA expression by 80%. Protein lysates from normal, fibrotic and MRC5 fibroblasts treated for 36 hours with TNF-α (10/ng/ml), IFN-γ (50 U/ml) or with both cytokines were then analyzed for PTPN13 expression. Western blot analysis showed that PTPN13 protein expression was not decreased in cells treated with IFN-γ alone, but PTPN13 expression decreased when the fibroblasts were treated with TNF-α. The decrease in PTPN13 protein expression was augmented when the cells were treated with both TNF-α and IFN-γ cytokines (FIG. 2B). To measure the kinetics of down-regulation of PTPN13 expression, a time course using both IFN-γ (50 U/ml) and TNF-α (10 ng/ml) was performed. Quantitative PCR demonstrated that PTPN13 mRNA levels began to decrease at 4 hours and continued to decrease up to 24 hour (FIG. 2C). Western blot analysis demonstrated that PTPN13 protein expression decreased at 18 hours with a maximal decrease at 24 hours (FIG. 2D). To determine if there was correlation between decreased PTPN13 protein expression and increased sensitivity to Fas-induced apoptosis, NHLF, FHLF and MRC5 cells were pretreated with TNF-α and IFN-γ for increasing amounts of time and subsequently activated with agonistic Fas antibody (250 ng/ml) for 2 hours (FIG. 2E). Pretreatment with TNF-α and IFN-γ had no effect on sensitivity to apoptosis up to the 18 hour time point. Fas stimulated caspase-8 activity was increased in lung fibroblasts that were pre-treated for 18 hours with TNF-α and IFN-γ. Longer pretreatment with TNF-α and IFN-γ showed a further increase in Fas-induced caspase-8 activity. These data show that normal and fibrotic primary lung fibroblasts have similar levels of expression of PTPN13 and respond in a similar manner to TNF-α and IFN-γ. There is an inverse correlation between the decrease in PTPN13 expression and increase in sensitivity to Fas-induced apoptosis, suggesting that PTPN13 blocks the apoptotic signaling cascade downstream of the Fas receptor.

### Example 2

### Decreased PTPN13 Expression Sensitizes Lung Fibroblasts to Fas-Induced Apoptosis

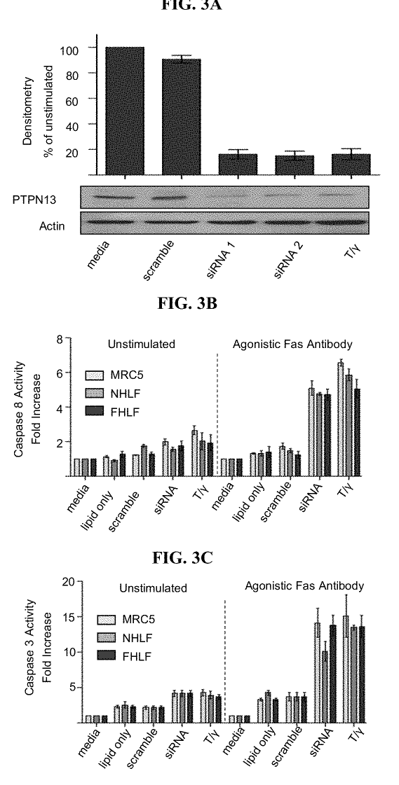

In one exemplary method, to assess whether PTPN13 expression prevents Fas-induced apoptosis, PTPN13 expression was knocked-down using siRNA. NHLF, FHLF and MRC5 cells were treated with siRNA (75 uM) directed toward the FERM domain of PTPN13. This siRNA knocks down (reduces the expression of) all four isoforms of PTPN13. After 36 hours of treatment with siRNA, PTPN13 expression was consistently decreased by >80% as determined by Western blot (see for example FIG. 3A). In FIG. 3A, the decrease in PTPN13 expression is equivalent between fibroblasts treated with specific PTPN13 siRNA and cells treated with both TNF-α and IFN-γ. To determine the effect of decreased PTPN13 expression on sensitization to Fas-induced apoptosis, normal non-diseased and MRC5 lung fibroblasts were stimulated with Fas-activating anti-Fas antibody (250 ng/ml) for 4 hours following pretreatment with siRNA or cytokines. Apoptosis was assayed by quantifying activation of caspase-3 and caspase-8 (FIG. 3C). Decreased expression of PTPN13 alone was not sufficient to induce caspase-3 or caspase-8 activation; however, specific knockdown of PTPN13 expression prior to Fas-ligation sensitized the lung fibroblasts to Fas-induced caspase-3 and caspase-8 activation. Knockdown of PTPN13 expression with siRNA in MRC5 lung fibroblasts increased the expression of Fas on the cell surface but did not increase the expression of Fas (FIG. 3D). These findings suggest that decreased expression of PTPN13 is sufficient to sensitize cell to Fas-induced apoptosis.

### Example 3

### Decreased PTPN13 Expression Leads to Formation of the Fas-Induced Death-Inducing Signaling Complex

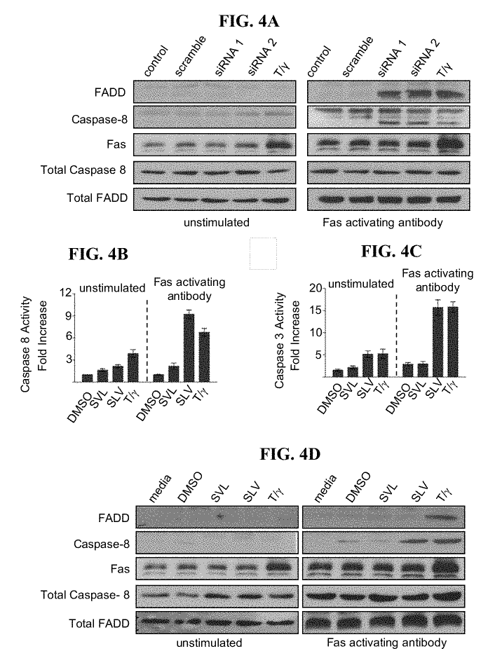

In death-receptor mediated apoptosis, formation of the DISC (Death-Inducing Signaling Complex) is essential for the initiation of apoptosis. Failure to form the Fas-induced DISC could lead to resistance to apoptosis in lung fibroblasts. Previous studies have shown that sensitization with TNF-α and IFN-γ increased recruitment of FADD and pro-caspase-8 to the Fas-induced DISC. To investigate whether decreased expression of PTPN13 promotes formation of the DISC, MRC5 cells were pretreated with PTPN13 siRNA or with TNF-α and IFN-γ as a positive control for 36 hours prior to stimulation with antagonistic Fas antibody. The cells were lysed, immunoprecipitated with anti-Fas antibody, and co-immunoprecipitated FADD and caspase-8 were detected by immunoblotting. FIG. 4A represents that FADD and active caspase-8 were not detected in anti-Fas immunoprecipitates of lung fibroblasts pretreated with PTPN13 siRNA but not stimulated with Fas. FADD and active caspase-8 were recruited to the DISC after Fas stimulation in lung fibroblasts pretreated with PTPN13 siRNA. To confirm these findings that the association of PTPN13 with Fas is likely responsible for blocking apoptosis, an inhibitory peptide was used to dissociate PTPN13 from Fas. PTPN13 binds to the extreme C-terminal end of Fas through a SLV tripeptide sequence in Fas 8. We added a cell soluble, acylated-SLV tripeptide to the lung fibroblasts for 2 hours prior to activating the cells with CH-11 activating Fas antibody (250 ng/ml) for 4 hours. When MRC5 lung fibroblasts were pretreated with the inhibitory acylated-SLV peptide, the fibroblasts were sensitized to Fas-induced apoptosis as demonstrated by an increase in caspase-8 and caspase-3 activity (FIG. 4B). Pretreatment with acylated-SVL and acylated-SLG control peptides did not increase Fas-induced caspase-8 or caspase-3 activity. These findings suggest that PTPN13 prevents recruitment of FADD to Fas. Decreased expression of PTPN13 alone was apparently not sufficient alone to induce recruitment of FADD to the DISC, suggesting that sensitization to Fas-induced apoptosis likely occurs in at least two steps. First, PTPN13 expression is decreased first then stimulation of the receptor can initiate the recruitment of FADD to Fas.

### Example 4

### Inhibition of Protein Tyrosine Phosphatase Activity Sensitizes Lung Fibroblasts to Fas-Induced Apoptosis

PTPN13 is a tyrosine phosphatase that has been demonstrated to associate with the C-terminal end of Fas. PTPN13 has been shown in astrocytoma cells to dephosphorylate Fas at tyrosine 291. Little is known about the regulatory function of tyrosine phosphorylation of Fas. In another exemplary embodiment, to assess the effect of TNF-α and IFN-γ sensitization on the levels of Fas tyrosine phosphorylation, MRC5 lung fibroblasts were treated with TNF-α and IFN-γ for increasing amounts of time. Because TNF-α is known to increase tyrosine phosphorylation of many proteins, such as MAP kinases and AKT, within minutes following stimulation, the kinetics of TNF-α and IFN-γ induced tyrosine phosphorylation of Fas were assessed. MRC5 lung fibroblasts were treated with TNF-α (10 ng/ml) and IFN-γ (50 U/ml) for up to 48 hours. The cells were lysed and analyzed by western blot for phosphorylated Fas, using a phospho-specific antibody for Fas phosho-Y291. TNF-α and IFN-γ increased tyrosine phosphorylation of Fas at tyrosine residue 291 beginning at 18 hours. There was no increase in phosphorylation of Fas at residue 291 prior to 6 hours (data not shown). The increase in tyrosine phosphorylation correlated with the decreased expression of PTPN13. To assess the effect of tyrosine phosphatase activity has on sensitivity to Fas-induced apoptosis, MRC5 lung fibroblasts were treated with pervanadate, a broad spectrum inhibitor of tyrosine phosphatases prior to Fas stimulation. MRC5 lung fibroblasts were treated with 150 μM pervanadate for 2 hours prior to simulating with Fas activating antibody (250 ng/ml) for 0 to 120 minutes. Pervanadate treatment increased caspase-8 activity 2-fold above untreated cells; however, adding Fas-activating antibody following the 2 hour pervanadate pre-treatment increased caspase-8 activity by 6-fold over unstimulated cells. The pervanadate treated MRC5 cells were lysed and analyzed the tyrosine phosphorylation of Fas by western blotting. Treatment of the lung fibroblasts with Fas-activating antibody alone did not increase the tyrosine phosphorylation of Fas at tyrosine 291. Pervanadate treatment increased tyrosine phosphorylation of Fas in a time dependent manner. To assess if decreased PTPN13 expression led to increased Fas phosphorylation, MRC5 lung fibroblasts were treated with PTPN13 siRNA for 36 hours. We then detected phosphorylated Fas by Western blot, using a phospho-specific antibody for Fas tyrosine residue 291. Phosphorylation of Fas tyrosine residue 291 was increased in lung fibroblasts pretreated with PTPN13 siRNA. SLV inhibitory peptide (10M) increased the amount of phosphorylation at tyrosine 291 on Fas suggesting that the inhibitory peptide dissociated PTPN13 from Fas, thereby inhibiting the ability of PTPN13 to dephosphorylate Fas. An increase in the basal tyrosine phosphorylation of the receptor is not sufficient to induce apoptosis; however, tyrosine phosphorylation of Fas at tyrosine 291 is required for sensitization to Fas-induced apoptosis.

### Example 5

### Deficiency of PTPN13 Leads to Decreased Bleomycin-Induced Fibrosis

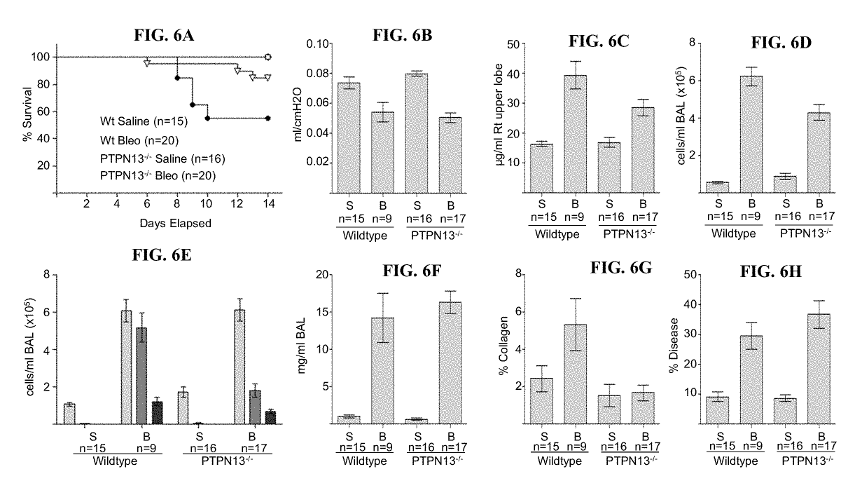

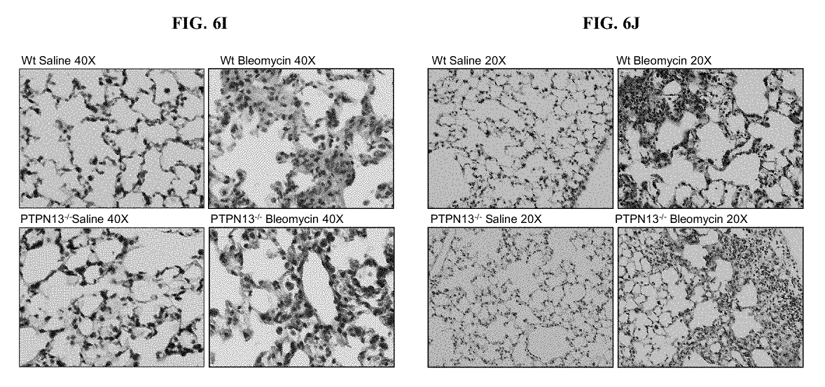

To assess the effect of PTPN13 on development of fibrosis in vivo, we used a murine model of fibrosis in a PTPN13−/− mouse. The PTPN13−/− mouse has been characterized by Grusby to have abnormal STAT4 signaling which leads to augmented skewing toward TH1 or TH2 phenotypes. 2.5 U/kg bleomycin was administered directly into the tracheas of PTPN13−/− mice along with wildtype controls. Mice were observed for two weeks for weight loss and other signs of sickness. Due to drastic weight decreases in the wildtype mice, two weeks was chosen as the end time point. Wildtype mice had a significant susceptibility to 2.5 U/kg bleomycin as demonstrated by increased mortality (see FIG. 6A). Of the 20 wildtype mice treated with bleomycin, 11 (55%) died before two weeks as compared to the PTPN13−/− mice where three of the 20 (15%) died. Saline had no effect on mortality. Then static compliance was measured. The saline treated wildtype and PTPN13−/− mice had similar values of static compliance. Both the bleomycin treated wildtype and PTPN13−/− mice had decreases in static compliance (FIG. 6b). Collagen content was measured in the lung using a hydroxyproline assay. Bleomycin treatment caused an increase in collagen in wildtype and PTPN13−/− mice as compared to the saline treated controls, however, the bleomycin treated PTPN13−/− mice have decreased collagen production as compared to the bleomycin treated wildtype mice (FIG. 6c). To determine a possible cause for the decreased static compliance in the PTPN13−/− mice, we also measured other markers of lung disease, such as cellular infiltration and capillary leak. We measured the cellularity of the bronchoalveolar fluid (BALF). Both wildtype and PTPN13−/− bleomycin treated mice had an increase in the total number of cells present in the BALF, however, the PTPN13−/− mice had a lower number of lymphocytes than the wildtype mice (FIG. 6D, 6E). Capillary leak was determined by albumin present in the BALF (FIG. 6F). Wildtype and PTPN13−/− mice had equivalent capillary leak, suggesting that 2.5 U/kg bleomycin causes similar injury in wildtype and PTPN13−/− lungs. To validate these findings, stereology was performed on lung sections to quantify presence of collagen and diseased areas. Stereology counting confirmed hydroxyproline data, showing that bleomycin treated wildtype mice have a significant increase in collagen deposition compared to PTPN13−/− mice (FIG. 6G). Percentage of diseased lung was also measured by stereology. Wildtype and PTPN13−/− mice treated with bleomycin had a significant increase in the percentage of lung involved with disease compared to the saline treated controls (FIG. 6H). Masson's trichrome staining of lung sections demonstrate that bleomycin treated wildtype lungs have increased collagen deposition as compared to PTPN13−/− lungs. Of particular interest, is the increase in cellularity in wildtype and PTPN13−/− samples, but the lack of collagen interspersed throughout the PTPN13−/− lungs (FIG. 6I, 6J). These findings suggest that 2.5 U/kg bleomycin causes significant injury in wildtype and PTPN13−/− lungs, however, this injury in PTPN13−/− does not lead to excessive collagen production and deposition as it does in the wildtype mice.

### FIGURES FURTHER DESCRIBED

FIG. 2: TNF-α and IFN-γ decrease PTPN13 expression. Normal human lung fibroblasts (NHLF) were harvested from healthy adult donors. Fibrotic human lung fibroblasts (FHLF) were harvested from IPF subjects. NHLF, FHLF and MRC5 lung fibroblasts were incubated in medium (0.1% serum) alone or were treated with 50 U/ml IFN-γ, 10 ng/ml TNF-α, or both cytokines for 36 hours. FIG. 2A. Top panel is a quantitative RT-PCR analysis of PTPN13 mRNA lower panel is a Western blot analysis for PTPN13 protein. FIG. 2B. Time course of PTPN13 mRNA expression following TNF-α (10 ng/ml) and IFN-g (50 U/ml) treatment. FIG. 2C. Western blot analysis of PTPN13 expression after MRC5, NHLF, and FHLF were treated with TNF-α (10 ng/ml) and IFN-g (50 U/ml) for times indicated. FIG. 2D. Time course of caspase-8 activation in MRC5 lung fibroblasts, NHLF and FHLF. Cells were treated with TNF-α (10 ng/ml) and IFN-g (50 U/ml) for indicated times, 0 to 48 hours. Fibroblasts were then stimulated with CH-11 Fas activating antibody (250 ng/ml) for 3 hours. Caspase-8 was measured using a fluorescence based assay.

FIG. 3: Decreased PTPN13 expression sensitizes fibroblasts to Fas-induced apoptosis. FIG. 3A. Specific siRNA decreases PTPN13 expression. NHLF, FHLF and MRC5 lung fibroblasts were treated with PTPN13 siRNA (75 mM), scrambled siRNA (75 mM) as a negative control or 50 U/ml IFN-γ and 10 ng/ml TNF-α as a positive control for 36 hours. PTPN13 expression was quantified by western blot analysis and densitometry was performed. FIG. 3B. NHLF, FHLF and MRC5 lung fibroblasts were incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α. Fas activating antibody (250 ng/ml) (Fas activating antibody) or PBS (unstimulated) was added for 2 hours before analyzing Caspase-8 activation by a fluorescence-based assay. FIG. 3C. NHLF, FHLF and MRC5 lung fibroblasts were incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α. Fas activating antibody (250 ng/ml) (Fas activating antibody) or PBS (unstimulated) was added for 2 hours before analyzing Caspase-3 activation by a fluorescence-based assay.

FIG. 4: Decreased expression of PTPN13 promotes formation of the Death Induced Signaling Complex (DISC) following Fas stimulation. FIG. 4A. MRC5 lung fibroblasts were incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α as a positive control prior to stimulation with 250 ng/ml activating Fas antibody for 4 hours. Cells were lysed and equal amounts of protein were immunoprecipitated with anti-Fas antibody. Co-precipitated caspase-8 and FADD were identified by western blot analysis. FIG. 4B. MRC5 lung fibroblasts were incubated in medium (0.1% serum) alone or were treated for 2 hours with acylated-SLV peptide (10 uM) or acylated-SVL peptide (10 uM) as a negative control. Treatment with IFN-γ (50 U/ml) and TNF-α (10 ng/ml) for 36 hours was used as a positive control. Fas activating antibody (250 ng/ml) (Fas activating antibody) or PBS (unstimulated) was then added for 2 hours before analyzing Caspase-8 activation by a fluorescence-based assay. FIG. 4C. MRC5 lung fibroblasts were incubated in medium (0.1% serum) alone or were treated with acylated-SLV peptide (10 uM) or acylated-SVL peptide (10 μM) as a negative control for 2 hours. Treatment with IFN-γ (50 U/ml) and TNF-α (10 ng/ml) for 36 hours was used as a positive control. Fas activating antibody (250 ng/ml) (Fas activating antibody) or PBS (unstimulated) was then added for 2 hours before analyzing Caspase-3 activation by a fluorescence-based assay. FIG. 4D. MRC5 lung fibroblasts were incubated in medium (0.1% serum) alone or treated with SLV (10 uM) for 2 hours. Treatment with SVL (10 uM) for 2 hours was used as a negative control and 50 U/ml IFN-γ and 10 ng/ml TNF-α for 36 hours was used as a positive control. The fibroblasts were then stimulated with activating Fas antibody (250 ng/ml) for 3 hours. Cells were lysed and equal amounts of protein were immunoprecipitated with anti-Fas antibody. Co-precipitated caspase-8 and FADD were identified by western blot analysis.

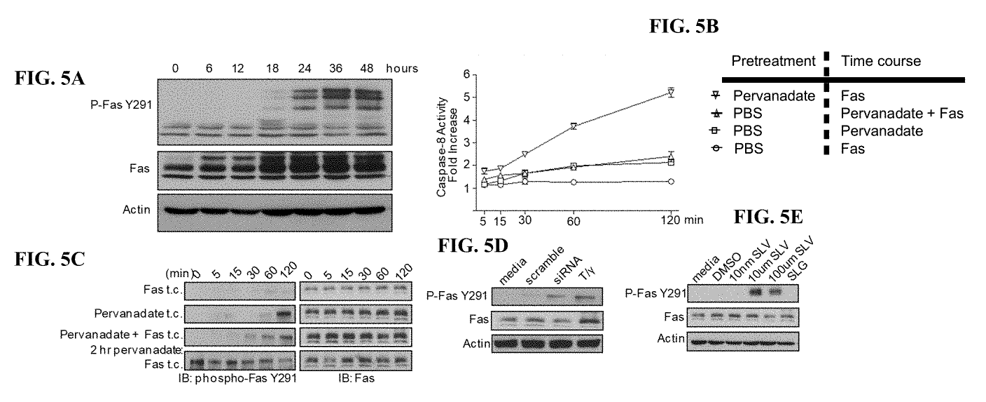

FIG. 5: Inhibition of tyrosine phosphatase activity sensitizes lung fibroblasts to Fas-induced apoptosis. FIG. 5A. Effects of TNF-α and IFN-γ on tyrosine phosphorylation of Fas. MRC5 lung fibroblasts were treated with TNF-α (10 ng/ml) and IFN-γ (50 U/ml) for indicated times. Western blot analysis of tyrosine phosphorylation of tyrosine 291 in Fas death domain using a phospho-specific anti-Fas Y291 antibody. FIG. 5B. Protein tyrosine inhibitor sensitizes lung fibroblasts to Fas-induced apoptosis. MRC5 lung fibroblasts were either preincubated with pervanadate (150 uM) or PBS as a control for 2 hours. Lung fibroblasts treated with PBS were then stimulated with activating Fas antibody (250 ng/ml), pervanadate (150 uM), activating Fas antibody (250 ng/ml) and pervanadate (150 uM) together for the times indicated. Lung fibroblasts pre-treated with pervanadate for 2 hours were then stimulated with activating Fas antibody for times indicated. Caspase-8 activation was measured by a fluorescence based assay. FIG. 5C. Protein tyrosine inhibitor increases tyrosine phosphorylation of tyrosine 291 on Fas. MRC5 lung fibroblasts were either pre-incubated with pervanadate (150 uM) or PBS as a control for 2 hours. Lung fibroblasts treated with PBS were then stimulated with activating Fas antibody (250 ng/ml), pervanadate (150 uM), activating Fas antibody (250 ng/ml) and pervanadate (150 uM) together for the times indicated. Lung fibroblasts pre-treated with pervanadate for 2 hours were then stimulated with activating Fas antibody for times indicated. Western blot analysis of tyrosine phosphorylation of tyrosine 291 in Fas death domain using a phospho-specific anti-Fas Y291 antibody. FIG. 5D. MRC5 lung fibroblasts were incubated in medium (0.1% serum) alone or were treated for 36 hours with PTPN13 siRNA (75 mM), scrambled siRNA (75 mM) as a negative control or both 50 U/ml IFN-γ and 10 ng/ml TNF-α as a positive control. Western blot analysis of tyrosine phosphorylation of tyrosine 291 in Fas death domain was performed using a phospho-specific anti-Fas Y291 antibody. FIG. 5E. MRC5 lung fibroblasts were incubated in medium (0.1% serum) alone or were treated with acylated-SLV peptide (10 uM) or acylated-SVL peptide (10 uM) as a negative control for 2 hours. Western blot analysis of tyrosine phosphorylation of tyrosine 291 in Fas death domain was performed using a phospho-specific anti-Fas Y291 antibody.

FIG. 6: Deficiency of PTPN13−/− Leads to Decreased Bleomycin-induced Lung Fibrosis. FIG. 6A. Survival curve of saline and bleomycin treated wildtype and PTPN13−/− mice. Mice had normal saline or 2.5 U/kg bleomycin directly administered to the lungs via the trachea. Mice were observed for two weeks for weight loss. Any mice having greater than 20% weight loss were euthanized. FIG. 6B. Measurement of static compliance. Mice were given pentabarbitol and pancuronium to paralyze the diaphragm before being placed on a flexi-Vent small animal ventilator. FIG. 6C. Collagen production is measured indirectly by hydroxyproline. The upper right lobe was digested overnight. FIG. 6D. Increased cell numbers in BALF. FIG. 6E. Differential of BALF. Macrophages, lymphocytes and neutrophils were counted from cytospin. FIG. 6F. Capillary leak in murine lungs. Albumin was measured by ELISA present in BALF. FIG. 6G. Collagen content as measured by stereology. Lungs were cut into 2 mm sections prior to being randomly embedded in paraffin. Three 5 micron sections from each lung were stained with Masson's trichrome. In a blinded, random method, five images of each slide were taken for stereology counting. FIG. 6H. Measurement of disease by stereology.

### Materials and Methods

Recombinant human TNF-α and IFN-γ were purchased from R&D Systems. Agonistic anti-human Fas antibody (clone CH-11) was obtained from Upstate Biotechnology. Anti-human Fas and anti-human FADD antibodies were obtained from Santa Cruz Biotechnology. Anti-caspase-8 antibody was obtained from Cell Signaling Technologies. Anti-PTPN13 antibody was purchased from R&D Systems.

### Cell Culture

The MRC5 cell line, a human fetal lung fibroblast cell line, was obtained from the ATCC and maintained in Dulbecco's minimal essential medium (DMEM) supplemented with 100 U/ml penicillin, 100 μg/ml L-glutamine, and 10% heat-inactivated fetal calf serum. Primary cultures of normal human lung fibroblasts were derived from non-diseased human lungs. Normal lung samples were subjected to the criteria as previously described and primary cultures of human fibrotic lung fibroblasts 3.

### Quantitative PCR

Total RNA was obtained from 1×106 MRC5 cells using the Qiagen RNA Miniprep Kit (Qiagen) following the manufacturer's instructions. The purified RNA was reverse transcribed using the Impromp-II Reverse Transcription Kit (Promega) and specific PCR products were generated using primers for PTPN13 (Applied Biosystems) and the Taqman Gene Amplification System (Applied Biosystems). The fluorescence values of the threshold cycles were collected and normalized to GAPDH. Data are expressed as the relative change in mRNA expression compared to the untreated control.

### Western Blotting Analysis

MRC5 cells were harvested and lysed in RIPA buffer (100 mM Tris, pH 7.4, 150 mM NaCl, 1% NP-40, 1% SDS, 0.5% sodium deoxycholate and 1 mM EDTA). Protein content of the lysates was determined by using the Bradford reagent (BioRad). Cell lysates (50 μg protein) were separated by SDS-PAGE on a 5-15% gradient acrylamide gel. Blocking and incubations with antibody were performed in TBS, 0.5% Tween-20 containing 5% milk. Blots were treated with enhanced chemiluminescence (Amersham International) followed by exposure to film.

### Apoptosis Assays

Active caspase-3 and caspase-8 were determined by using the Caspase-Glo Kits (Promega) as per manufacturer's instructions. Luminescence was measured with a (brand) luminometer. Data are expressed as fold change over unstimulated control.

### Co-Immunoprecipitation

MRC5 cells were lysed in NP-40 lysis buffer (50 mM Tris-HCl, pH 7.2, containing 150 mM NaCl, 10% glycerol, 1% NP-40, 1 mM phenylmethysufonyl fluoride, 10 μg/ml leupeptin, 5 g/ml aprotinin, and 1 mM Na3VO4) and centrifuged at 14,000 rpm for 10 min at 4° C. to obtain postnuclear supernatants. Three hundred and fifty μg of protein were pre-cleared with anti-goat IgG Sepharose beads (ebioscience). Fas was then immunoprecipitated with goat anti-human Fas antibody or non-immune goat IgG as a control for 24 h at 4° C. Immune complexes were then pulled down with anti-goat IgG Sepharose beads. The beads were washed four times in lysis buffer, boiled in Laemlli sample buffer and resolved by electrophoresis through 12% SDS-acrylamide gels. Detection of specific proteins was by Western blot analysis.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

1.-11. (canceled)

12. A kit for treating a subject having a pulmonary disorder comprising:
at least one container; and
at least one agent capable of modulating PTPN13 comprising one or more cytokine compositions.

13. A method for treating a subject having cancer comprising:
administering a therapeutically effective amount of an agent capable of associating with PTPN13 to the subject and modulating PTPN13 expression or activity in the subject, wherein modulating the PTPN13 expression or activity in the subject treats the cancer in the subject.

14. The method of claim 13, wherein the cancer comprises pancreatic, stomach, ovarian, lung, melanoma or colon cancer.

15. The method of claim 13, wherein the agent is administered directly to a tumor of the subject.

16. The method of claim 13, wherein the agent reduces cancer cells in the subject by inducing apoptosis in the cancer cells.

17. A method for reducing scarring in a subject in need thereof, the method comprising: administering a therapeutically effective amount of an agent capable of associating with PTPN13 to the subject and modulating PTPN13 expression or activity in the subject, wherein modulating the PTPN13 expression or activity in the subject reduces scarring in the subject.

18. The method of claim 17, wherein administration to the subject is before, during or after undergoing plastic surgery, a wound healing process or recovering from a transplant or graft.

19. The method of claim 17, wherein the subject is a burn patient.

20. A method for modulating tyrosine phosphatase in a subject in need thereof, comprising administering to the subject a therapeutically effective amount of a composition capable of associating with the tyrosine phosphatase and modulating tyrosine phosphatase expression or activity in the subject.

21.-30. (canceled)
